(19) United States
(12) Patent Application Publication
(10) Pub. No.: US 2008/0245375 A1
(43) Pub. Date: Oct. 9, 2008

(54) Benign Prostatic Hyperplasia Treatments

(75) Inventor: Julie Trudel, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/697,190

(22) Filed: Apr. 5, 2007

(51) Int. Cl. A61B 19/00

(52) U.S. Cl. 128/898; 604/500

(57) ABSTRACT

Disclosed herein are methods to treat benign prostatic hyperplasia (BPH) using bioactive-material eluting medical devices.

### FIELD OF THE INVENTION

The present invention relates to the use of bioactive-material eluting medical devices in the treatment of benign prostatic hyperplasia (BPH).

### BACKGROUND OF THE INVENTION

Benign Prostatic Hyperplasia (BPH) is a nonmalignant enlargement of the prostate gland that afflicts more than 10 million adult males in the United States, and many millions more throughout the rest of the world. It is estimated that over 50% of men over the age of 60 have BPH with the number reaching 90% of men over the age of 85. BPH leads to a number of problems including urinary tract obstruction and other related urinary tract symptomology.

Currently, two general classes of orally administered bioactive materials are used to treat the symptoms of BPH. One class includes compounds that inhibit the production of the active form of testosterone (dihyrdotestosterone or DHT). Systemic use of these bioactive materials, however, can cause a loss of libido and loss of muscle mass and tone in males and is associated with an increased occurrence of high grade prostate cancer. In addition, this therapy is limited by the very long delay (months) between the first administration of the bioactive material and any significant reduction in prostate size. The second class of bioactive materials currently used to treat BPH are a adrenergic blockers which relax the smooth muscles, allowing urine to pass through the urethra more freely. While this class of bioactive materials reduces symptoms more quickly than the first, it does not reduce the size of the prostate or prevent it from growing larger.

BPH can also be treated by open prostatectomy, transurethral resection of the prostate (TURP), transurethral microwave therapy (TUMT), laser therapy, transurethral needle ablation (TUNA), transurethral incision of the prostate (TUIP), prostatic stents and water induced thermotherapy (WIT). Thus, while there are a number of treatment options available, none satisfactorily treats or prevents the recurrence of BPH. Therefore, there is room for improvement in the treatment of this disease.

### SUMMARY OF THE INVENTION

The present invention provides a new treatment for benign prostatic hyperplasia (BPH). Specifically, the present invention includes a method of locally treating BPH comprising positioning a bioactive-material eluting medical device at a treatment site. At least one bioactive material elutes from the medical device at a rate that achieves a prophylactically effective amount and/or a therapeutically effective amount of the bioactive material at the treatment site. In one embodiment, the treatment site will include a site of urethral narrowing.

Bioactive materials used in accordance with the present invention can include compounds that are one or more of an a blocker, an inhibitor of 5-α-reductase, an α-adrenergic agonist, an α-adrenergic antagonist, an antiandrogen, an antiestrogen, an aromatase inhibitor, a 17β-hydroxysteroid dehydrogenase (17β-HSD) inhibitor, a LHRH agonist, an LHRH antagonist, an anti-proliferative, an mTOR inhibitor, and combinations thereof.

In certain embodiments, the bioactive materials will elute from a polymer. Polymers used in accordance with the present invention can have a characteristic selected from the group consisting of degradable, nondegradable, biodegradable, nonbiodegradable, bioerodible, nonbioerodable, bioadsorbable, nonbioadsorbable, controlled release, noncontrolled release, removable, nonremovable, and nonincompatible combinations thereof.

In one embodiment, the methods according to the present invention will include positioning a bioactive material eluting medical device at a treatment site to treat BPH in combination with another treatment to treat BPH wherein the second treatment is selected from the group consisting of the administration of oral medication, open prostatectomy, transurethral resection of the prostate (TURP), transurethral microwave therapy (TUMT), laser therapy, transurethral needle ablation (TUNA), transurethral incision of the prostate (TUIP), water induced thermotherapy (WIT) and combinations thereof.

### BRIEF DESCRIPTION OF THE DRAWINGS

FIG. 1 depicts the effects of rapamycin on proliferation of human prostatic stromal cells according to the teachings of the present invention.

FIG. 2 depicts the effects of paclitaxel on proliferation of human prostatic stromal cells according to the teachings of the present invention.

FIG. 2 depicts the effects of terazosin on proliferation of human prostatic stromal cells according to the teachings of the present invention.

### DEFINITION OF TERMS

Prior to setting forth the invention, it may be helpful to an understanding thereof to set forth definitions of certain terms that will be used hereinafter:

Administering or Administration Of: As used herein, “administering” or “administration of” a bioactive material to a subject (and grammatical equivalents of this phrase) includes both direct administration, including self-administration, and indirect administration, including the act of prescribing a bioactive material. For example, as used herein, a physician who instructs a patient to self-administer a bioactive material and/or provides a patient with a prescription for a bioactive material is administering the bioactive material to the patient.

Bioactive Material(s): As used herein “bioactive material” shall include any agent having a therapeutic effect in an animal. Exemplary, non limiting examples include a bioactive material can be a protein, a polypeptide, a polysaccharide (e.g. heparin), an oligosaccharide, a mono- or disaccharide, an organic compound, an organometallic compound, or an inorganic compound. It can include a living or senescent cell, bacterium, virus, or part thereof. It can include a biologically active molecule such as a hormone, a growth factor, a growth factor producing virus, a growth factor inhibitor, a growth factor receptor, an anti-inflammatory agent, an antimetabolite, an integrin blocker, or a complete or partial functional insense or antisense gene. It can also include a man-made particle or material, which carries a biologically relevant or active material. An example is a nanoparticle comprising a core with a drug and a coating on the core.

Bioactive materials also can include drugs such as chemical or biological compounds that can have a therapeutic effect on a biological organism. Exemplary, non limiting examples include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, anti-angiogenic agents, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers of the present invention.

Furthermore, bioactive materials can also include a blockers, inhibitors of 5-α-reductase, α-adrenergic agonists, α-adrenergic antagonists, antiandrogens, antiestrogens, aromatase inhibitors, 17β-hydroxysteroid dehydrogenase (17β-HSD) inhibitors, LHRH agonists, LHRH antagonists, mTOR inhibitors and combinations thereof.

Bioactive materials also can include precursor materials that exhibit the relevant biological activity after being metabolized, broken-down (e.g. cleaving molecular components), or otherwise processed and modified within the body. These can include such precursor materials that might otherwise be considered relatively biologically inert or otherwise not effective for a particular result related to the medical condition to be treated prior to such modification.

Combinations, blends, or other preparations of any of the foregoing examples can be made and still be considered bioactive materials within the intended meaning herein. Aspects of the present invention directed toward bioactive materials can include any or all of the foregoing examples.

Prophylactically Effective Amount: As used herein, a “prophylactically effective amount” of a bioactive material is an amount of a bioactive material that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose, and can occur only after administration of a series of doses. Thus, a prophylactically effective amount can be administered in one or more administrations.

Reduction: As used herein, “reduction” of a symptom or symptoms (and grammatical equivalents of this phrase) means decreasing the severity or frequency of symptom(s), or eliminating the symptom(s).

Stents: As used herein, the term “stents” refers to devices that are used to maintain patency of a body lumen, including those of the urologic system.

Therapeutically Effective Amount: As used herein, a “therapeutically effective amount” of a bioactive material is an amount of a bioactive material that, when administered to a subject with BPH, will have the intended therapeutic effect, e.g., alleviation, amelioration, palliation or elimination of one or more manifestations of BPH in the subject. The full therapeutic effect does not necessarily occur by administration of one dose, and can occur only after administration of a series of doses. Thus, a therapeutically effective amount can be administered in one or more administrations.

Treating: As used herein, “treating” a condition or patient refers to taking steps to obtain beneficial or desired results, including clinical results. For purposes of this invention, beneficial or desired clinical results include, but are not limited to, alleviation or amelioration of one or more symptoms of BPH, diminishment of the extent of the disease, the delay or slowing of disease progression, amelioration, palliation or stabilization of the disease state, and other beneficial results described herein.

### DETAILED DESCRIPTION OF THE INVENTION

The prostate is a walnut-sized gland that forms part of the male reproductive system. The gland consists of several lobes, or regions, enclosed by a dense fibrous capsule. It is located between the bladder and the rectum and wraps around the urethra, the tube that carries urine out from the bladder through the penis. There are generally three glandular zones in a prostate gland: central, peripheral and transitional. The transitional zone is located right behind the place where the seminal vesicles are merging with urethra. This transitional zone tends to be predisposed to benign enlargement.

The prostate reaches its normal size and weight (about 20 grams) soon after puberty. The size and weight of the prostate typically remain stable until the individual reaches his mid-forties. At this age, the prostate typically begins to enlarge through a process of excessive cellular proliferation, often leading to benign prostatic hyperplastia (BPH). This overgrowth can occur in both smooth muscle and glandular epithelial tissues and has been attributed to a number of different causes, including hormones and growth factors as well as generally to the aging process. As an indication of the potential severity of this disease, while the prostate gland measures approximately 20 cc in young, healthy males, it can grow to over 200 cc, a greater than 10-fold increase, in men affected by BPH.

Benign prostate hyperplasia can cause distressing urination symptoms. As the disease progresses the dense capsule surrounding the enlarging prostate prevents it from further expansion outward and forces the prostate to press against the urethra, partially obstructing urine flow. The tension in the smooth muscles of the prostate also increases which causes further compression of the urethra and reduction of urine flow. Some symptoms of BPH stem from the gradual loss of bladder function leading to an incomplete emptying of the bladder. These symptoms can include straining to urinate, a weak or intermittent stream, an increased frequency of urination, pain during urination, and incontinence—the involuntary loss of urine following an uncontrollable sense of urgency. These symptoms alone can negatively affect the quality of life of affected men. Left untreated, BPH can cause even more severe complications, such as urinary tract infection, acute urinary retention, and uremia.

While there are treatment options available for BPH, including oral medications, open prostatectomy, transurethral resection of the prostate (TURP), transurethral microwave therapy (TUMT), laser therapy, transurethral needle ablation (TUNA), transurethral incision of the prostate (TUIP), prostatic stents and water induced thermotherapy (WIT), none of these available treatment options satisfactorily treat or prevent the recurrence of BPH. Therefore, there is room for improvement in the treatment of this disease.

The present invention provides a novel approach to the treatment of BPH. Specifically, the present invention provides for the local delivery of bioactive materials that inhibit the growth of prostatic tissue. In one embodiment, a bioactive material-eluting medical device is implanted at a treatment site to locally target prostatic enlargement. Medical devices suitable for implantation at a treatment site in the prostate include, but are not limited to stents, pellets, meshes, patches, and strips. In one embodiment of the present invention, urethral stents are placed at urethral narrowings to enlarge urethral strictures caused by BPH and to help maintain the patency of the urethra. In another embodiment, bioactive material-eluting pellets are placed within the prostate to controllably release a bioactive material at the treatment site. The bioactive material eluted from the device will allow for prostatic tissue to become quiescent, and for adequate healing of the urethra after stent deployment. This will lead to better outcome of BPH intervention and the use of bioactive material-eluting medical devices could lead to a significant improvement in the treatment of BPH.

Prostatic/urethral medical devices used in accordance with the present invention can be either permanent or temporary devices. Non-limiting examples of appropriate stents that can be used in accordance with the present invention include the UroLume endoprosthesis from American Medical Systems, Inc. and cobalt alloy-based Driver® stents (Medtronic Inc., Minneapolis, Minn.). Stents used in accordance with the present invention will generally have a diameter of at least about 2.5 mm. Certain stents used in accordance with the present invention can be degradable or nondegradable, bioadsorbable or nonbioadsorbable and/or removable or nonremovable.

The bioactive material-eluting pellets can take a wide variety of forms and may be fashioned from a polymeric carrier, including for example, rods, particles, beads, or capsules. The bioactive material and the polymeric carrier may form a homogeneous matrix, or the bioactive material may be encapsulated in some way within the polymer. In their simplest form, the bioactive agent-eluting pellets consist of a dispersion of the bioactive material in a polymer matrix. The bioactive material is typically released as the polymeric matrix biodegrades in vivo into soluble products that can be excreted from the body. The bioactive material may be first encapsulated in a microsphere and then combined with the polymer in such a way that at least a portion of the microsphere structure is maintained. Alternatively, the bioactive material may be sufficiently immiscible in the polymer that it is dispersed as small droplets, rather than being dissolved, in the polymer. Either form is acceptable, but it is preferred that, regardless of the homogeneity of the composition, the release rate of the bioactive material in vivo remains controlled.

In another embodiment of the present invention, bioactive materials can be delivered to the treatment site in a biocompatible solution and injected locally into the target tissue using an injection catheter or other means known to persons of ordinary skill in the art.

A wide variety of bioactive materials can be delivered to the external portion of the body passageway, or to smooth muscle cells via the adventia of the body passageway within or adjacent to the prostate gland. For example, in one embodiment, a needle or catheter is guided into the prostate gland adjacent to the urethra via the transrectal route (or alternatively transperineally) under ultrasound guidance and through this deliver a bioactive material. In alternative embodiments, the bioactive materials are injected in several quadrants of the gland and/or around the urethra. In another embodiment, a catheter is guided to the treatment site via the urethra and through this deliver a bioactive material. The needle or catheter can also be placed under direct palpation or under endoscopic, fluoroscopic, CT or MRI guidance, and administered at intervals. As an alternative, the placement of pellets via a catheter or trocar can also be accomplished. The above procedures can be accomplished alone or in conjunction with a stent placed in the prostatic urethra. By avoiding urethral instrumentation or damage to the urethra, the sphincter mechanism would be left intact, avoiding incontinence, and a stricture is less likely.

In another embodiment of the present invention, the bioactive materials can be injected directly into prostate tissue using an injection catheter or other in situ injection means. In another embodiment, a radiofrequency transurethral needle ablation device can be used for the local injection of bioactive materials.

A variety of bioactive materials can be released from the devices of the present invention to treat BPH. Classes of bioactive materials that can be especially useful include, without limitation, α blockers, inhibitors of 5-α-reductase, α-adrenergic agonists, α-adrenergic antagonists, antiandrogens, antiestrogens, aromatase inhibitors, 17β-hydroxysteroid dehydrogenase (17β-HSD) inhibitors, and leutenizing hormone releasing hormone (LHRH) agonists, LHRH antagonists, anti-proliferatives, inhibitors of the mammalian target of rapamycin (mTOR), and any combination of these classes.

Representative non-limiting examples of compounds within these classes include (compounds may fall under more than one class): (i) 5-α-reductase inhibitors: terazosin (Hytrin® (Abbott Laboratories, Corp., Abbott Park, Ill.)), doxazosin (Cardura® (Pfizer, Inc., New York, N.Y.)), tamsulosin (Flomax® (Yamanouchi Pharmaceutical Co., Tokyo, JP)), Merck L 652,931, dutasteride (DUAGEN® (Glaxo Group, Ltd., Greenford Middlesex, UK), (5α,17β)-N-[2,5-bis(trifluoromethy)phenyl]-2-oxo-4-azaandrost-1-ene-17-carboximide; 4-azasteroid compounds generally, the 4-azasteroid compounds described in Liang et al., J. Biol. chem. 259: 734-739, 1984 and Brooks et al., Steroids 47: 1-19, 1986); finasteride (available under the trade name PROSCAR® (Merck & Co., Inc., Whitehouse Station, N.J.; chemical name: (5α,17β)-N-(1,1-dimethylethy)-3-oxo-4-azaandrost-1-ene-17-carb-oxamide). 4-azasteroid compounds described in U.S. Pat. Nos. 4,220,735; 4,377,584; and 4,760,071; N,N-diethyl-4-methyl-3-oxo-4-aza-5α-androstane-17β-carboxamide (4-MA); 6-methylene-4-pregnene-3,20-dione (LY 207320) (as described by Toomey et al., Proc. 71st Annual Meeting of Endocr. Soc., abst. #1226, p. 329, 1989); MK-906 (a product of Merck, Sharp & Dohme (Australia); 17β-N,N-diethylcarbamoyl-4-methyl-4-aza-5α-androstan-3-one (4-MA) (Brooks et al., Endocrinology 109: 830, 1981; Liang et al., Endocrinology 112: 1460, 1983); 6-methylene-4-pregnene-3,20-dione (Petrow et al., J. Endocrinol. 95: 311-313, 1982); 4-methyl-3-oxo-4-aza-5α-pregnane-30(s) carboxylate (Kadohama et al., J. Natl. Cancer Inst. 74: 475-486; 1985); PNU 157706, [N-(1,1,1,3,3,3-hexafluorophenyl-propyl)-3-oxo-4-aza-5α-androst-1-e-ne-17β-carboxamide] (see diSalle, et al., J Steroid Biochem. Mol. Biol. 64, 179 (1998)); PNU 157706; and herbal supplement saw palmetto; (ii) α-adrenergic agonists: clonidine and naphazoline; (iii) α-adrenergic antagonists: prazosin, terazosin and phenoxybenzamine; (iv) antiandrogens: cyproterone acetate; megestrol acetate; medroxyprogesterone acetate; chlormadinone acetate; WIN 49596; bicalutamide (CASODEX® (AstraZeneca, Wilmington, Del.; (±)N-[4-cyano-3-(trifluoromethy) phenyl]-3-[(4-fluoropheny) sulfonyl]-2-hydroxy-2-methyl propanimide; described in U.S. Pat. No. 4,636,505); an R-enantiomer of bicalutamide; flutamide; nilutamide; RU 58642; RU 58841; and the antiandrogens described in U.S. Pat. No. 3,875,229; U.S. Pat. No. 4,097,578; U.S. Pat. No. 4,239,776; U.S. Pat. No. 4,386,080; U.S. Pat. No. 5,994,362; and U.S. Pat. No. 5,872,150; (v) antiestrogens: tamoxifen; and (vi) inhibitors of 17β-hydroxysteroid dehydrogenase: N-butyl, N-methyl-11-(16′α-chloro-3′,17′β-dihydroxy estra-1′,3′,5′(10′)-trien-7′α-yl)undecanamide (EM 139); N-n-butyl-N-methyl-11-(16′α-chloro-3′,17′α-dihydroxy-estra-1′,3′,5′(10′)-trien-7′α-yl)undecananmide (EM 170); and N-n-butyl-N-methyl-11-(16′α-bromo-3′,17α-dihydroxy-estra-1′,3′, 5′(10′)-trien-7′α-yl) undecanamide (EM 171). Ionidamine or Ionidamine analogs as described in U.S. Pat. No. 6,989,400 may also be useful as a bioactive material in accordance with the teachings of the present invention. All the above references are incorporate herein in their entirety for all they contain regarding bioactive materials.

Anti-proliferatives include, but are not limited to, cytostatic agents such as, but not limited to, paclitaxel and macrolide antibiotics including FKBP-12 binding compounds, such as, but not limited to sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent Ser. No. 10/930,487) and zotarolimus (ABT-578; see U.S. Pat. Nos. 6,015,815 and 6,329,386).

U.S. Pat. No. 5,753,641 reports a method of treating BPH using a combination of a 5-α-reductase, such as finasteride, and an α1-adrenoreceptor antagonist, such as terazosin. A method of treating BPH with a combination of an α1-adrenoreceptor antagonist, such as terazosin, and saw palmetto extract is reported in U.S. Pat. No. 6,200,573. Treatment of BPH by the administration of the non-steroidal antiandrogen bicalutamide is reported by Eri, et al., Urology 56, 261 (2000). Further, U.S. Pat. No. 6,423,698 describes administering a combination of two or more compounds selected from the group consisting of an inhibitor of 5-α-reductase activity, an anti-estrogen, an inhibitor of aromatase activity, an inhibitor of 17β-hydroxysteroid dehydrogenase activity and, in some cases, an anti-androgen and/or an LHRH agonist. All the above references are incorporate herein in their entirety for all they contain regarding bioactive materials.

U.S. Pat. No. 4,472,382 discloses treatment of BPH with an anti-androgen and certain peptides which act as LHRH agonists. U.S. Pat. No. 4,596,797 discloses aromatase inhibitors as a method of prophylaxis and/or treatment of prostatic hyperplasia. U.S. Pat. No. 4,760,053 describes a treatment of certain cancers which combines an LHRH agonist with an antiandrogen and/or an antiestrogen and/or at least one inhibitor of sex steroid biosynthesis. U.S. Pat. No. 4,659,695 discloses a method of treatment of prostate cancer in susceptible male animals including humans whose testicular hormonal secretions are blocked by surgical or chemical means, e.g. by use of an LHRH agonist, which comprises administering an antiandrogen, e.g. flutamide, in association with at least one inhibitor of sex steroid biosynthesis, e.g. aminoglutethimide and/or ketoconazole. The bioactive materials disclosed in the aforementioned references alone or in combination are all appropriate for use in accordance with the bioactive material-eluting stents of the present invention. All the above references are incorporate herein in their entirety for all they contain regarding bioactive materials.

Numerous polymer systems can be appropriate for use as bioactive material carriers for the bioactive materials of the present invention so long as the polymer achieves adequate bioactive material release to the surrounding tissue. The polymers can be degradable or nondegradable and/or bioadsorbable or nonbioadsorbable. The polymers can provide for controlled or non-controlled bioactive material release. Ideally, the polymers that are used will remain intact while the device is maneuvered in and positioned within the urethra and be compatible with widely used sterilization methods.

A listing of polymers that can be used in accordance with the present invention include rapidly bioerodible polymers such as, without limitation, poly[lactide-co-glycolide], polyanhydrides, and polyorthoesters, whose carboxylic groups are exposed on the external surface as their smooth surface erodes. In addition, polymers containing labile bonds, such as, without limitation, polyanhydrides and polyesters can also be used. Representative natural polymers that can be used include, without limitation, proteins, such as zein, modified zein, casein, gelatin, gluten, serum albumin, or collagen, and polysaccharides, such as, without limitation, cellulose, dextrans, polyhyaluronic acid, polymers of acrylic and methacrylic esters and alginic acid. Representative synthetic polymers that can be used in accordance with the present invention include, without limitation, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Synthetically modified natural polymers that can be used in accordance with the present invention include, without limitation, alkyl celluloses, hydroxyalkyl celluloses, cellulose ethers, cellulose esters, and nitrocelluloses. Other polymers that can be used in accordance with the present invention include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol. Representative bioerodible polymers include polylactides, polyglycolides and copolymers thereof, poly(ethylene terephthalate), poly(butic acid), poly(valeric acid), poly(lactide-co-caprolactone), poly[lactide-co-glycolide], polyanhydrides, polyorthoesters, blends and copolymers thereof.

These described polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

### EXAMPLE 1

In an non-limiting example of coating a metallic stent with a polymer and a bioactive material, a solution of a suitable polymer and a bioactive material (for example ABT-578 in a polymer:drug ratio of 70:30 by weight) in chloroform was sprayed on a stent and allowed to dry producing a controlled release coating on the stent. The release of drug from the stent into a solvent is measured by high performance liquid chromatography (HPLC).

### EXAMPLE 2

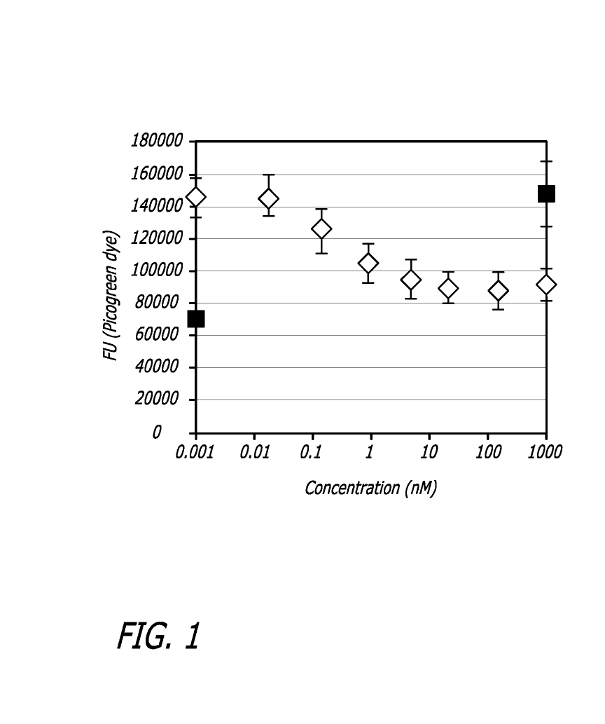

Human prostatic cells are sensitive to anti-proliferative agents such as inhibitors of the mammalian target of rapamycin (mTOR) such as rapamycin and ABT-578 and pro-apoptotic agents such as paclitaxel (FIGS. 1-3).

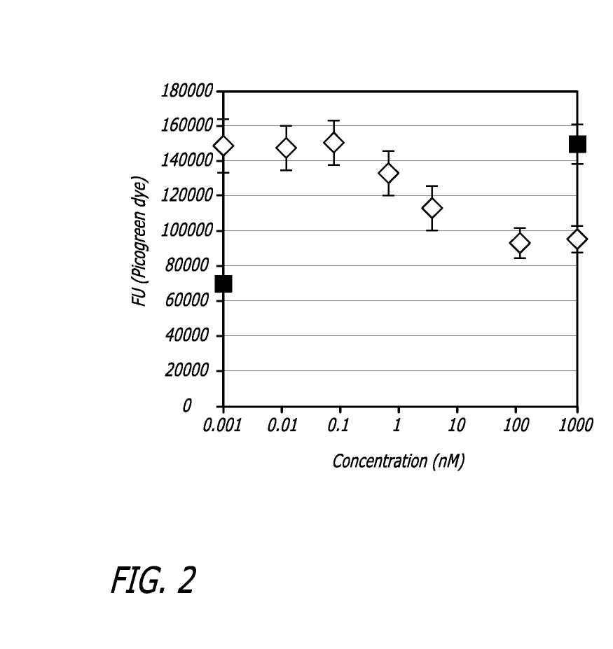

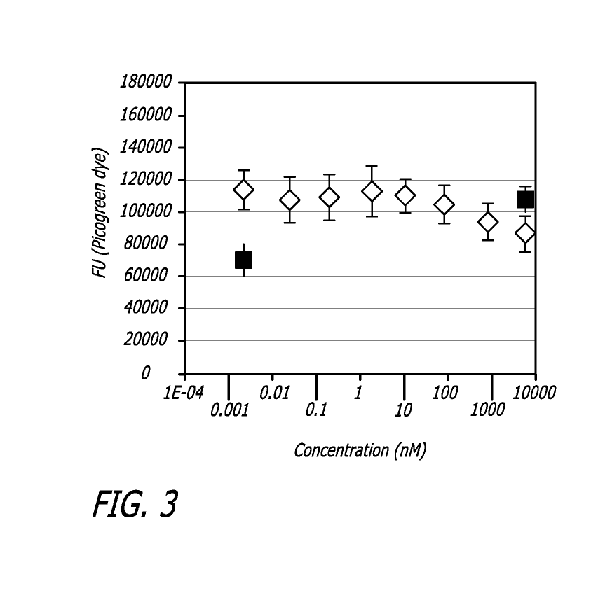

Human prostatic stromal cells (HPrSC) were grown in prostatic cell growth medium and basal medium to 70% confluency. The cells were then cultured for 48 hours under a variety of conditions. One group of cells was growth arrested (baseline cell number represented by square symbol in lower left of graph in FIGS. 1-3) and a second group of cells was grown in culture medium without anti-proliferative agents in the presence of transforming growth factor β1 (maximum cell growth represented by square symbol in upper right of graph in FIGS. 1-3). Additional cells were cultured in the presence of increasing concentrations of rapamycin (FIG. 1), paclitaxel (FIG. 2) and terazosin (FIG. 3).

After 48 hours the cell proliferation in each culture was quantified by measuring the amount of double-stranded DNA by uptake of picogreen dye. The amount of double-stranded DNA in a culture is proportional to cell number.

Both rapamycin (FIG. 1) and paclitaxel (FIG. 2) inhibited the proliferation of human prostatic stromal cells.

### EXAMPLE 3

In this study, the bioactive materials Cardura® (Pfizer, Inc., New York, N.Y.) and Hytrin® (Abbott Laboratories, Corp., Abbott Park, Ill.) are evaluated in patients with benign prostatic hyperplasia (BPH).

The study will include nine treatment groups consisting of males between the ages of 60 and 70 presenting for the treatment of BPH: (1) no treatment control; (2) oral medication (Cardura®); (3) oral medication (Hytrin®); (4) bare metal stent control; (5) polymer coated stent control; (6) Cardura® fast-eluting stent; (7) Cardura® slow-eluting stent; (8) Hytrin® fast-eluting stent; and (9) Hytrin® slow-eluting stent.

Patients self-evaluate the severity of their symptoms on a scale of 1 to 10 over a 6 month time period and also rate the presence and severity of any side effects on a scale of 1 to 10. The results will demonstrate at least that patients receiving a bioactive-material eluting stent will show as much improvement as those receiving oral medications while reporting fewer and/or less sever adverse side effects.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that can be employed are within the scope of the invention. Thus, by way of example, but not of limitation, alternative configurations of the present invention can be utilized in accordance with the teachings herein. Accordingly, the present invention is not limited to that precisely as shown and described.

What is claimed is:

1. A method of treating benign prostatic hyperplasia (BPH) comprising positioning a bioactive material eluting medical device at a treatment site, wherein said medical device elutes at least one bioactive material at said treatment site.

2. A method according to claim 1 wherein said bioactive material eluting medical device elutes an amount of at least one bioactive material that constitutes a prophylactically effective amount and/or a therapeutically effective amount.

3. A method according to claim 1 wherein said treatment site is a urethral narrowing.

4. A method according to claim 1 wherein said medical device is selected from the group consisting of stents, pellets, catheters, needles, and transurethral needle ablation devices.

5. A method according to claim 4 wherein said medical device comprises a stent.

6. A method according to claim 4 wherein said medical device comprises pellets.

7. A method according to claim 1 wherein said at least one bioactive material is selected from a class of compounds selected from the group consisting of a blockers, inhibitors of 5-α-reductase, α-adrenergic agonists, α-adrenergic antagonists, antiandrogens, antiestrogens, aromatase inhibitors, 17β-hydroxysteroid dehydrogenase (17β-HSD) inhibitors, LHRH agonists, LHRH antagonists, anti-proliferatives, mTOR inhibitors and combinations thereof.

8. A method according to claim 7 wherein said bioactive material is an mTOR inhibitor.

9. A method according to claim 1 wherein said bioactive material elutes from a polymer.

10. A method according to claim 9 wherein said polymer has a characteristic selected from the group consisting of degradable, nondegradable, biodegradable, nonbiodegradable, bioerodible, nonbioerodable, bioadsorbable, nonbioadsorbable, controlled release, noncontrolled release, removable, nonremovable, and nonincompatible combinations thereof.

11. A method according to claim 1 wherein said treating occurs in combination with another therapy selected from the group consisting of the administration of oral medication, open prostatectomy, transurethral resection of the prostate (TURP), transurethral microwave therapy (TUMT), laser therapy, transurethral needle ablation (TUNA), transurethral incision of the prostate (TUIP), water induced thermotherapy (WIT) and combinations thereof.
